(19) United States
(12) Patent Application Publication
(10) Pub. No.: US 2003/0124149 A1
(43) Pub. Date: Jul. 3, 2003

(54) Bioactive absorbable microparticles as therapeutic vaccines

(76) Inventors: Shalaby W. Shalaby, Anderson, SC (US); Waleed S.W. Shalaby, Philadelphia, PA (US); Edward Y. Woo, Plymouth Meeting, PA (US); Heidi Yeh, Gladwyne, PA (US); Carl H. June, Merion Station, PA (US)

Correspondence Address: LEIGH P. GREGORY, ATTORNEY AT LAW, PO BOX 168, CLEMSON, SC, 29633-0168, US

(21) Appl. No.: 10/191,013

(22) Filed: Jul. 8, 2002

Related U.S. Application Data
(60) Provisional application No. 60/303,417, filed on Jul. 6, 2001.

(51) Int. Cl. A61K 39/00; A61K 9/50

(52) U.S. Cl. 424/277.1; 424/501

(57) ABSTRACT

A therapeutic vaccine for tumor immunotherapy is disclosed which comprises absorbable microparticles/nanoparticles capable of delivering immobilized stimulatory/costimulatory signals and releasing necessary cytokines to promote an anti-tumor effect. The microparticles/nanoparticles are formed of an absorbable crystalline polymer, preferably acid-terminated polyglycolide. Other bioactive agents such as tumor specific antigens, DNA or RNA may be immobilized of the surface of the particles.

The present application claims priority to earlier filed provisional application, U.S. Serial No. 60/303,417, filed Jul. 6, 2001.

### BACKGROUND OF THE INVENTION

Tumor immunotherapy involves the treatment of cancer through manipulation of the immune system. The strategy of immunomodulation may be more efficacious and less toxic than standard chemotherapy or radiation therapy. However, the modest success from recent clinical trials suggests that a number of issues have yet to be resolved. It is generally agreed that multiple components must co-exist to establish an effective, antigen-specific anticancer immune response. First, there must be appropriate presentation of tumor-specific antigens to cytotoxic (cd8+) and helper (cd4+) T-lymphocytes. Antigen presentation must also be accompanied by costimulation in order to expand a naive precursor T cell population. The efficacy of clonal expansion is markedly enhanced by professional antigen presenting cells (e.g. dendritic cells) which express MHC-restricted antigen and necessary costimulatory molecules. Local cytokine production is also expected to play a critical role to promote chemotaxis of other important immune cells and support T cell activation/expansion. Without the aforementioned components, an effective anti-tumor response may not be appreciated.

A large body of in formation has emerged supporting the use of various stimulatory molecules and cytokines to elicit antitumor responses. Multiple strategies have shown promise in the area of peptide specific vaccines, ex-vivo activation/expansion of T cells or tumors, and tumor cell or bystander cell transfections with various expression vectors. However, the time, cost, and patient morbidity involved with tumor/bystander cell procurement, modification, and maintenance could limit the application of immunotherapy to a small, select group of patients. Furthermore, the lack of immunogenic, tumor-specific antigens and the potential array of cytokines required for adequate responses could also limit broad application of this technology.

A related prior art is the use of cation-exchangers as part of controlled drug delivery systems for binding peptide, proteins, and bioactive agents which was disclosed by one of the inventors (Shalaby: U.S. Pat. Nos. 5,612,052; 5,714,159; pending U.S. Ser. No. 09/015,394, filed Jan. 19, 1998). However, using a synergistic combination of bioactive agents, as described in the present invention, has not been described in the prior art.

### SUMMARY OF THE INVENTION

Recognizing the potential shortcomings of current immunotherapy, the inventors conceived of a non cell-based approach to generate tumor-specific T cell responses by combining stimulatory and costimulatory signals with necessary cytokines. The invention herein describes the use of novel biodegradable, microparticles that deliver stimulatory (anti-CD3)/co-stimulatory (anti-CD28) signals to activate T cells and the controlled release of cytokines (granulocyte-macrophage colony stimulating factor, GM-CSF) to bias T cell proliferation toward tumor-specific T cells.

The use of polyglycolide microparticles as therapeutic vaccines offers a versatile means to introduce an array of macromolecules and cytokines to the tumor site that may be critical to eliciting an immunologic response against cancer. There are also numerous practical advantages related to ease of preparation, modification, storage, biodegradability, and existing approval for clinical use by the FDA. In the clinical setting, employing these systems near or at the time of diagnosis and/or debulking when tumor burden is lowest may improve response and decrease patient morbidity. In short, the potential for rapid and less costly implementation of new immunologic advances to a broader range of patients is a distinct possibility using this technology. Ultimately, this could prove to be a practical alternative to current cell-based strategies.

Thus, in one aspect the present invention is directed to a therapeutic vaccine for treating cancer which is based on absorbable microparticles/nanoparticles formed of a crystalline absorbable polymer having a heat of fusion of at least about 60 Joules/gram, wherein the microparticles/nanoparticles have a surface with at least one bioactive agent immobilized thereon. Preferably, the surface of the microparticles/nanoparticles is anion-forming. It is also preferred that the bioactive agent is ionically bound to the surface of the microparticles/nanoparticles.

In one preferred embodiment the crystalline absorbable polymer is an acid-bearing polyglycolide, preferably an acid-terminated polyglycolide.

Generally, the surface of the microparticles/nanoparticles is microporous/nanoporous.

In a preferred embodiment anti-CD3/anti-CD28 and GM-CSF are ionically bonded to the surface of the microparticles/nanoparticles.

In another preferred embodiment there is at least one bioactive agent ionically bonded to the surface of the microparticles/nanoparticles, which is selected from the group consisting essentially of anti-CTLA-4, anti-ICOS, 4-1BB ligand, IL-12, IL-18, IL-2, and IFN-gamma.

Optionally, a polycationic molecule, such as polylysine, may be chemically bonded to the surface of the microparticles/nanoparticles. In such case, anti-CD3/anti-CD28 and GM-CSF may be bound to the polycationic molecule or at least one bioactive agent, which is selected from the group consisting essentially of anti-CTLA-4, anti-ICOS, 4-1BB ligand, IL-12, IL-18, IL-2, and IFN-gamma may be bound to the polycationic molecule. Preferably, the polycationic molecule is ionically bonded to the surface of the microparticles/nanoparticles, although it may be at least partially covalently bonded thereto.

In another embodiment the surface of the microparticles/nanoparticles is cation-forming. Such may be the case when the crystalline absorbable polymer is, for example, an amine-bearing polyester. Hereagain, the bioactive agent or agents may be ionically bonded to the cation-forming surface.

### BRIEF DESCRIPTION OF THE FIGURES OF THE DRAWING

FIG. 1 illustrates the preparation of microparticles bearing granulocyte-macrophage colony stimulating factor in accordance with the present invention;

FIG. 2A illustrates the activation of resting human t-cells by anti-human CD3/CD28-bearing microparticles in accordance with the present invention;

FIG. 2B illustrates the activation of resting mouse t-cells by anti-mouse CD3/CD28-bearing microparticles in accordance with the present invention;

FIG. 3 illustrates the release of granulocyte-macrophage colony stimulating factor from GMCSF-microparticles prepared from polyglycolide and polyglycolide microparticles having polylysine chemically bonded thereto; and

FIG. 4 illustrates the Growth of TF-1 cells cultured with granulocyte-macrophage colony stimulating factor-releasing microparticles (●), soluble GM-CSF @ 2 ng/ml (♦), uncoated polyglycolide microparticles (▴), and plain culture media (▪).

### DESCRIPTION OF THE PREFERRED EMBODIMENT

One broad aspect of this invention deals with the use of absorbable nanoparticles and/or microparticles carrying covalently linked carboxylic or amine groups on their surfaces, wherein such surfaces comprise a nanoporous and/or microporous texture that is capable of immobilizing one or more protein or peptide through physicochemical interaction. The chemical interaction takes place through ionic or covalent bonding, while the physical interaction can prevail through hydrophobic binding or simple physical entrapment. The absorbable microparticles/nanoparticles of this present invention comprise absorbable chains which are, in turn, made of one or more of the following types of repeat units linked with hydrolyzable bonds—ester, ether-esters, carbonates, anhydrides, and amides.

The bioactive agents of the present invention can be synergistic combinations of one (or more) peptide, polypeptide, or protein, such as granulocyte-macrophage colony stimulating factor (GM-CSF), interleukin 12 (IL-12), and interleukin 2 (IL-2); stimulatory molecules that bind to the T cell receptor CD3 (anti-CD3), or costimulatory molecules that bind to cytotoxic T lymphocyte-associated protein-4 (CTLA-4), inducible co-stimulator (ICOS), or 4-1BB. This invention also deals with antigen and vaccine immobilization on microparticle or nanoparticle surfaces, such as tumor-specific or associated antigens, DNA, and RNA.

Another aspect of this invention deals with the use of a crystalline acid-bearing polyester having a heat of fusion of at least 60 J/g and preferably, more than 80 J/g, and more preferably more than 100 J/g, such as acid-terminated polyglycolide microparticles (PG-MP) as versatile carriers that could be surface-modified for either immobilization of stimulatory moieties or cytokine release. The adsorption affinity for macromolecules on polymeric surfaces is primarily controlled by the degree of hydrophobic interactions. Thus, in the context of proteins, the extent of irreversible adsorption is proportional to the molar mass. A shift from reversible to predominantly irreversible adsorption can conceivably occur at a critical molar mass above which larger macromolecules would remain immobilized on the microparticle surface (e.g. immunoglobulins) and below which desorption would occur (e.g. cytokines). Adsorption affinity may also be manipulated through electrostatic interactions that favors charge neutralization. In this scenario, the microparticle surface charge or zeta potential could be modified based on the isoelectric point (pI) of the protein to increase cytokine loading. This can be illustrated, in part, by polyglycolide microparticles surface-modified with anti-CD3/anti-CD28 and GM-CSF as presented in this invention. For these and similarly activated microparticles or nanoparticles, acid-terminated polyglycolide microparticles were prepared to have an anionic charge on the surface to modulate protein adsorption/desorption by hydrophobic and electrostatic interactions.

Thermodynamically, interactions between proteins and polymer surfaces range from highly specific antibody associations with particular epitopes to relatively weaker, non-specific electrostatic or hydrophobic binding. In the latter case, two limiting situations of reversible and irreversible adsorption can arise. Irreversible adsorption occurs when the contact energy between peptide constituents and the surface is larger than the thermal energy. Adsorption is characterized by surface spreading and a flattening of the configuration to maximize the number of contacts. As a result, late arriving proteins find few sites to adsorb and are bound loosely. This results in a bimodal distribution of long-lived irreversibly and reversibly bound proteins. However, it is likely that multiple adsorbate conformations exist and that the probability of adsorption may also depend on the formation of highly heterogeneous population of adsorbate clusters. Adsorption would not only depend on excluded volume interactions, but also on the strength of attractive interactions between adsorbate molecules. Thus, adsorption affinity may also depend on the relative contribution from negative cooperativity, independent or Langmuir-type behavior, and positive cooperativity. Regardless of the theoretical model, the data presented in this invention show that relatively high molecular weight immunoglobulins (anti-cd3 and anti-cd28) will predominantly undergo irreversible adsorption. Although the antibodies likely adopt a flattened conformation, the net effect is maintenance of biologic activity as evidenced by T cell activation. Previous experiments conducted by the inventors highlighted the critical importance of co-stimulation for activation of resting T cells. However, the current method of cd3/cd28-MP preparation employs equal concentrations of the two antibodies as a bidispersed system. Thus, differences in individual binding affinities may lead to preferential adsorption of either antibody. Although, the two immunoglobulins are similar in size, a competitive adsorption phenomena exists which may depend on the aforementioned factors.

The use of novel microparticles for cytokine release in combination with other synergistic molecules emphasizes the versatility of these systems as general carriers. The observation of GM-CSF release relates to significant and sustained reversible adsorption. Reversible adsorption occurs when the net energy of individual peptides is smaller than the thermal energy. If the energy gained by the peptide is small, local relaxation of the protein structure is relatively fast leading to rapid equilibration. Most theories underlying reversible adsorption would predict that desorption is a relatively slow process requiring the simultaneous release of all anchored contacts which is energetically unfavorable. The initial rapid release of GM-CSF and the presence of surface-bound proteins after release indicates that multiple adsorbate conformations exist yielding a distribution of reversibly and irreversibly bound GM-CSF. Furthermore, reversibly bound GM-CSF conformations maintain biologic activity as evidenced by growth studies using a GM-CSF-dependent cell line (TF-1). Modifying the surface charge by introducing poly-1-lysine increases the relative adsorption affinity of GM-CSF via charge neutralization. It is conceived that the interaction between PG and poly-1-lysine is relatively strong given the negative zeta potential on the surface and the positive charge on the protein. Based on the ESCA (electron spectroscopy for chemical analysis) data, however, the relatively low nitrogen level for the PG-Lys indicates a moderate level of adsorption. With this in mind, it is conceivable that repulsive interactions between lysine peptides produces a relatively rigid structure that is conformationally limited with respect to adsorption. Thus, it is likely that bare areas exist on the PG-Lys surface that would favor GM-CSF adsorption. Adsorption, however, is more energetically favorable due to electrostatic interactions between the poly-1-lysine and GM-CSF. Despite the apparent limited adsorption of poly-1-lysine, ESCA results suggest a nearly three-fold increase in GM-CSF, which would support the contribution of electrostatic interactions. The increase in early GM-CSF release or reversible adsorption reflects the increased binding affinity for the PG-Lys surface. However, a surprising observation was the sustained release of GM-CSF observed out to 26 days. It is conceived that the GM-CSF remaining on the PG-Lys surface after the initial release of cytokine resembles an adsorbate conformation that favors irreversible adsorption. Over time, the degradation of microparticle releases anchored peptides until desorption becomes energetically favorable. Thus, the later release profile resembles a degradation-controlled mechanism. The important observation here is the recognition of GM-CSF by ELISA after 26 days in PBS at 37° C. This suggests that the proportion of more strongly bound adsorbate exists as a conformationally active form that was maintained for the duration of the release experiment. This active form of GM-CSF may have accounted for the majority of reversibly adsorbed cytokine. Most importantly, however, is that, according to the present invention, sustained release of GM-CSF can be achieved using a simple process of reversible adsorption as opposed to more conventional microencapsulation techniques. One additional aspect of this invention deals with measures to improve GM-CSF binding, through covalently linking polycationic structures on the PG surface. Furthermore, the unique advantage of these systems, subject of this invention, is the ease to which it can be applied to other potential immunomodulating agents. This may include antibodies directed at costimulatory molecules such as CTLA-4, ICOS, and 4-1BB, cytokines such as IL-2 and IL-12, or even tumor specific antigens.

This invention emphasizes non cell-based, combination immunotherapy directed at different arms of the immune response. Recently, Hurwitz et al. (Hurwitz A A, Yu T F, Leach D R, Allison J P, CTLA-4 blockade synergizes with tumor-derived granulocyte-macrophage colony-stimulating factor for treatment of an experimental mammary carcinoma, PNAS, 1998, 95 (17), 10067-10071) used a combination CTLA-4 blockade and a GM-CSF-expressing tumor vaccine to reject SM1 tumors. They found that the combination resulted in regression of parental SM1 tumors, despite the ineffectiveness of either treatment alone. This synergistic therapy resulted in long-lasting immunity to SM1 tumors, which depended on both CD4⁺ and CD8⁺ T cells. These findings were also reproduced in the poorly immunogenic murine melanoma B16-BL6 (van Elsas A, Hurwitz A A, Allison J P. Combination immunotherapy of B16 melanoma using anti-cytotoxic T lymphocyte-associated antigen 4 (CTLA-4) and granulocyte/macrophage colony-stimulating factor (GM-CSF)-producing vaccines induces rejection of subcutaneous and metastatic tumors accompanied by autoimmune depigmentation. J. Exp. Med. Aug. 2, 1999; 190(3):355-66). In this work, investigators observed 80% eradication of established tumor in 80%, whereas each treatment by itself showed little or no effect. Mice surviving a primary challenge rejected a secondary challenge with B16-BL6 or the parental B16-F0 line. This same strategy elicited an effective response against primary prostate tumors in a transgenic mouse model (Hurwitz A A, Foster B A, Kwon E D, Truong T, Choi E M, Greenberg N M, Burg M B, Allison J P. Combination immunotherapy of primary prostate cancer in a transgenic mouse model using CTLA-4 blockade. Cancer Res. May 1, 2000; 60(9):2444-8). The results of the tumor prevention experiments, described in the present invention, are consistent with the preceding observations in that combination immunotherapy was superior to the individual components alone. In contrast, however, each component. as described in this invention, elicits significant antitumor properties independently. Although the release of GM-CSF from polymeric systems described in the prior art has shown mixed efficacy depending on the tumor model (Egilmez N K et al., Cytokine immunotherapy of cancer with controlled release biodegradable microspheres in a human tumor zenograft/SCID mouse model. Cancer Immunol Immunother. March 1998; 46(1):21-4). In situ tumor vaccination with interleukin-12-encapsulated biodegradable microspheres: induction of tumor regression and potent antitumor immunity. Cancer Res. Jul. 15, 2000;60(14):3832-7), the present invention describes the first animal model showing that anti-cd3/cd28 microparticles can elicit an antitumor response. In combination, the effect is synergistic. This invention also deals with a specific, novel PG-microparticles system with immobilized bioactive agents that prevent mouse tumor implantation. The finding can be extended further to the prevention of tumor implantation and/or regression in humans.

The results of the two tumor prevention experiments in mice are shown in Table I. Dissection of the flanks showed that the microparticles formed a well-circumscribed conglomerate in the subcutaneous tissue. This was seen with all control and treatment groups using the 56 micron microparticles. However, a dramatic decrease in mechanical integrity was noted with the 7 micron microparticles. Upon removal, they had a viscoelastic consistency in contrast to the 56 μm particles, which maintained their shape. This dramatic level of degradation was not surprising given the increased surface area to hydrolyze the polymer chains. However, the microparticle systems displayed no intrinsic ability to impair tumor implantation. Average tumor sizes of 8.2 mm and 8.6 mm were found in the tumor only and microparticle control groups, respectively. Significant efficacy was observed when Meth A cells were co-injected with either the cd3/cdD28-MP or GMCSF-MP alone. Only 5/16 mice developed tumor when Meth A cells were co-injected with the cd3/cd28-MP alone. This also corresponded to a nearly 86% reduction in average tumor diameter. Mice receiving GMCSF-MP alone developed tumor in only 2/8 mice. Again, a nearly 86% reduction in average tumor diameter was seen. The most significant effect was seen when the cd3-cd28-MP and GMCSF-MP were combined. There was no evidence of tumor implantation with either the 56 micron (0/16) or 7 micron (0/8) microparticle sizes. The concern over microparticle size was prompted by consistent early findings in the vaccine literature showing that microspheres less than 26 μm in size are taken up by the reticuloendothelial system. Furthermore, phagocytosis is more efficient for microparticles less than 10 μm in size. Thus, phagocytosis of cd3/cd28-MP and/or GMCSF-MP could potentially compromise efficacy and possibly cause to T cell anergy. However, results, subject of this invention, surprisingly failed to show any compromising efficacy as a function of particle size. Interestingly, much of the data of the prior art pertaining to microparticle phagocytosis was generated using slowly degraded materials based on polylactide-co-glycolide copolymers. In contrast, the PG-MP of the present invention undergo significant degradation leading to the formation of soft aggregates after two weeks. Degradation was more pronounced with the 7 μm PG-MP, which exhibited a viscoelastic consistency and underwent aggregation. This in vivo microparticle aggregation may have made the 7 μm systems equally as effective as the inherently larger particles. The latter are considered to be less practical to use than the easily injectable 7 μm system.

TABLE 1
Results of Mouse Tumor Prevention Experiments Using Meth A Fibrosarcoma Cells

| | Number of | Mice with tumor | Average Tumor | |
|---|---|---|---|---|
| Group | mice | present | Diameter (mm) | SE (+/−) |
| Tumor only | 16 | 16 | 8.2 | 0.71 |
| Microparticle control | 15 | 15 | 8.6 | 1.10 |
| Cd3/cd28-MP alone | 16 | 5 | 1.2 | 0.46 |
| GMCSF-MP alone | 8 | 2 | 1.0 | 0.68 |
| Combination group | 16 | 0 | — | — |
| (56 μm particle size) | | | | |
| Combination group | 8 | 0 | — | — |
| (7 μm particle size) | | | | |

More details of the present invention are provided but not limited to the examples given below.

### EXAMPLE 1

### Preparation of Polyglycolide Microparticles (PG-MP)

Acid-terminated PG microparticles were prepared by Poly-Med, Inc. (Anderson, S.C.) as previously described (U.S. Pat. No. 5.653,992). Briefly, glycolic acid, glycolide, and stannous octoate catalyst were mixed in a dry flask equipped with a magnetic stirrer under a dry nitrogen atmosphere. The reactants were slowly heated to 100° C. under agitation for approximately 20 min. When the reaction mixture opacified, the temperature was increased to 160° C. The reaction was maintained at 160° C. to achieve high conversion. After cooling, the reaction product was isolated and quenched with liquid nitrogen; the resulting solid PG was dried in a vacuum oven at 35° C. overnight. The PG was ground to a fine powder using a Jet-mill and/or Wiley mill depending on the required particle size. The particles were then size separated with differential sieving screens. The residual monomer was extracted using anhydrous acetone at 25° C. The size distribution was determined using a particle size analyzer (Accusizer Particle Sizing Systems, Inc., Santa Barbara, Calif.). Characterization of the microparticle surface consisted of scanning electron microscopy (SEM), potentiometric titration of the accessible carboxylic acid groups, and electron spectroscopy for chemical analysis (ESCA). The net surface charge or zeta potential was evaluated using an electroacoustic spectrometer to measure the colloid vibration current (CVI) at a frequency of 1 Mhz. Microparticles were dispersed in phosphate-buffered saline (PBS, pH-7.2) for CVI measurements. Selected samples were also assayed for the presence of endotoxin to eliminate confounding antitumor responses that could be potentially observed in planned animal experiments. The endotoxin assay was determined using a chromogenic limulus amebocyte lysate test kit.

### EXAMPLE 2

### Preparation of Anti-cd3/cd28 Microparticles (cd3/cd28-MP)

Anti-human CD3/CD28-MP and anti-mouse cd3/cd28-MP were prepared by incubating PG-MP in phosphate-buffered saline (PBS, pH-7.2) containing either 2 μg/ml, 5 μg/ml, or 10 μg/ml of the respective antibodies. Mouse anti-human CD3 (OKT-3) was purchased from Ortho Biotech (Raritan, N.J.) and hamster anti-mouse cd3 mAb was generously provided by B. Blazar (University of Minnesota, Minneapolis, Minn.). Anti-human CD28 mAb (mAb9.3) and hamster anti-mouse cd28 mAb was provided by one of the inventors. Samples were incubated on a rotating wheel for 18 hrs at 37° C. and then washed in PBS. ELISA experiments were done to determine the level of reversibly bound antibody due to concerns that antibody desorption could lead to T cell anergy. Ten mg samples of anti-cd3/cd28-MP were placed in Eppendorf® vials containing 1 ml of PBS. They were incubated on a rotating wheel at 37° C. for seven days. Microparticles were centrifuged daily to remove all PBS followed by addition of fresh PBS. Samples were analyzed by ELISA using an HRP-conjugated anti-hamster IgG cocktail (Armenian and Syrian, B D Pharmingen, San Diego, Calif.). Data reported reflect the average optical density of duplicate wells (corrected for background) converted to ng/ml of antibody by linear regression analysis of standard curves run with each assay. The detection limit was 750 pg/ml.

### EXAMPLE 3

### Preparation of GM-CSF-Microparticles (GMCSF-MP)

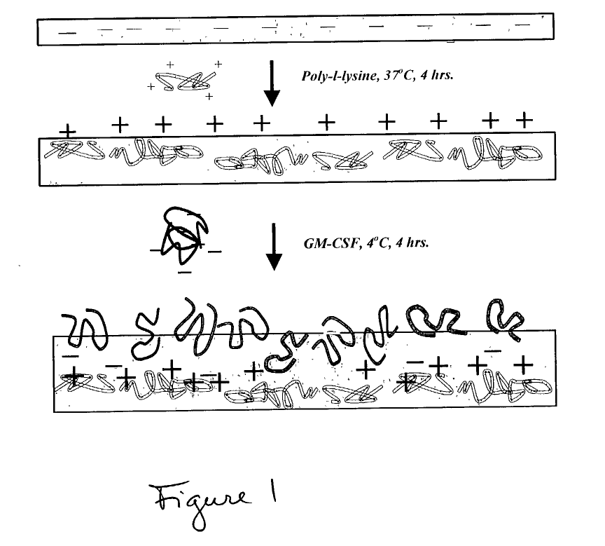

GMCSF-MP was prepared as is illustrated in FIG. 1. PG-MP was first incubated in PBS containing 5 mg/ml of poly-1-lysine (MW ˜70,000; Sigma, St. Louis, Mo.) for 4 hrs at 37° C. The intent was to shift the zeta potential to a more positive surface charge through charge neutralization given that the isoelectric point (pI) of human-GM-CSF is 5.26, and thus, would require a more positively charged surface to improve adsorption affinity. PG/Lys microparticles were then washed once in PBS and incubated with human-GM-CSF (PG-Lys/GMCSF) (Leukine, Immunex, Seattle, Wash.) at 100 μg/ml for 4 hrs at 4° C. Previous experiments determined that this was optimal for GM-CSF adsorption. After GM-CSF loading, the samples were washed in PBS and stored at 4° C. prior to use. PG-MP were also loaded with h-GM-CSF in the absence of poly-1-lysine as a control (PG-GMCSF).

FIG. 1 illustrates the preparation of GM-CSF microparticles in PBS. PG-MP were first incubated in poly-1-lysine (5 mg/ml) for 4 hrs at 37° C. to alter the surface charge. After washing in PBS, the PG/Lys-MP were incubated with GM-CSF (pI-5.26) for 4 hrs at 4° C. GMCSF-MP were then washed once in and stored at 4° C. prior to each study.

### EXAMPLE 4

### T Cell Activation by Anti-cd3/cd28 Microparticles

Activation of resting T cells was studied using both the anti-human CD3/CD28-MP and anti-mouse cc3/cd28-MP. Human peripheral blood lymphocytes were isolated by ficoll gradient centrifugation from leukopacks obtained by apheresis of healthy donors. CD4+ T cells were purified as previously described using a negative selection method with magnetic beads (Dynal, Lake Success, N.Y.). Mouse T cells were collected from the spleens of 6-8 week old female Balb-Cl mice. Splenic cells were isolated by forced perfusion with PBS followed by suspension in ACK lysing buffer. T cell enrichment was achieved by a negative selection method using rat-anti-mouse cd14 (Pharmingen, San Diego, Calif.), cd11b (Pharmingen, San Diego, Calif.) and B220 (B-cell marker, D. Allman, University of Pennsylvania, Philadelphia, Pa.) monoclonal antibodies. Sheep anti-rat magnetic beads (Dynal, Lake Success, N.Y.) were then added to remove antibody-bound cells.

Human CD4+ cells (0.1×10⁶ cells) or were added to 96 well flat-bottom plates and cultured in RPMI 1640 (BioWhittaker, Walkersville, Md.) supplemented with 10% FCS (Hyclone, Logan, Utah) and 2 mM 1-glutamine (BioWhittaker). Cells were incubated in triplicate with either 0.6 mg/well of PG-MP as a control or anti-CD3/CD28-MP prepared with 2 μg/ml, 5 μg/ml, or 10 μg/ml of antibody. Anti-CD3/CD28 magnetic beads (Dynal, Lake Success, N.Y.). were used as a positive control. Previous studies have shown that a 3:1 bead to cell ratio is optimal for T cell activation. Resting mice T cells (0.1×10⁶ cells) studied in a similar fashion using PG-MP as a control and the anti-cd3/cd28-MP prepared with 2 μg/ml, 5 μg/ml, or 10 μg/ml of antibody. Positive controls consisted of well plates coated with anti-cd3 (10 μg/ml) and anti-cd28 (10 μg/ml) for 18 hrs at 37° C. Proliferation was assessed by pulsing the plates with 2 μCi/well [³H]TdR on day 3 followed by cell harvesting 18 hours later. Day three was chosen for pulsing the T cells based on previous data with human T cells showing maximum proliferation between day three and four. Detection of radioactivity from cell free supernatant was performed on a Trilux® liquid scintillation counter (Perkin-Elmer Wallac, Gaithersburg, Md.). Data reported reflects the average of three wells/condition.

### EXAMPLE 5

### Flow Cytometric Analysis of T Cells Activated by Anti-cd3/cd28-MP

Two-color flow cytometric analysis was performed to determine intracellular cytokine production from mouse T cells cultured with either PG-MP or anti-cd3/cd28-MP. Cells were removed on day two of culture, washed once in FACS buffer (PBS 0.05% FCS, 2 mM EDTA, 0.01% sodium azide), and surface stained with FITC-conjugated anti-cd3 mAb (B D Pharmingen, San Diego, Calif.). The cells were permeabilized for 10 minutes with FACS permeabilizing solution as per manufacturer's guidelines and stained for interferon-gamma (PE-conjugated α-IFN-γ; B D Pharmingen, San Diego, Calif.) and interleukin-4 (PE-conjugated α-IFN-IL4, B D Pharmingen, San Diego, Calif.) for 30 minutes in the dark at RT. Flow cytometry was performed on a Becton Dickinson FACS Calibur (San Jose, Calif.).

### EXAMPLE 6

### GM-CSF Release Studies

The release of h-GM-CSF from PG-GMCSF and PG-Lys/GMCSF microparticles was performed to assess the magnitude of reversibly adsorbed protein. Ten mg of respective microparticles were placed in Eppendorf® vials containing 1 ml of PBS. Release studies were carried out on a rotating wheel at 37° C. Microparticles were centrifuged daily to remove all PBS followed by addition of fresh PBS. Samples were then frozen at −20° C. prior to analysis. At the completion of the study, thawed samples were analyzed for GM-CSF by ELISA using a commercially available kit. (Quanktikine GM-CSF Immunoassay kit; R&D Systems, Minneapolis, Minn.)

### EXAMPLE 7

### Growth of TF-1 Cells by GMCSF-MP

The bioactivity of GM-CSF released from the PG-Lys/GMCSF microparticles was tested using a h-GM-CSF-dependent cell line. TF-1 cells (American Type Culture Collection, ATCC, Rockville, Md.) are a human lymphoblast line that completely depend on h-GM-CSF for long term growth. TF-1 cells were grown in RPMI 1640 (BioWhittaker, Walkersville, Md.) containing 10% FCS (Hyclone, Logan, Utah), 2 mM 1-glutamine (BioWhittaker), 1.5 g/l sodium bicarbonate, 10 mM HEPES (BioWhittaker), 1 mM sodium pyruvate (BioWhittaker). The culture media was supplemented with 2 ng/ml of h-GM-CSF. TF-1 cells were maintained at 1×10⁵ cells/ml by adding fresh media and h-GM-CSF every two days as recommended by the ATCC. Growth studies were performed by first washing the cells twice with h-GM-CSF-free media. TF-1 cells (1×10⁵ cells) were added to a 24-well culture plate and allocated to receive either no GMCSF, soluble h-GM-CSF (2 ng/ml), PG-MP (5, 10, or 20 mg/well), or PG-Lys/GMCSF (5, 10, or 20 mg/well). Cell cultures were counted on a daily basis using a Coulter Counter Multisizer II (Coulter, Hialeah, Fla.). TF-1 concentrations were maintained at 1×105 cells/ml by the addition of fresh media. TF-1 cells supplemented with soluble GM-CSF received additional cytokine to maintain a concentration of 2 ng/ml. However, TF-1 cultured with the PG-Lys/GMCSF did not receive any additional supplementation of either soluble GM-CSF or PG-Lys/GMCSF microparticles. Growth curves were carried out for two weeks.

### EXAMPLE 8

### Mouse Tumor Prevention Experiments

The efficacy of PG-cd3/cd28 and PG-Lys/GMCSF microparticles to prevent tumor growth in Balb-Cl mice (Taconic Farms, Inc., Germantown, N.Y.) was studied in a prevention model using a syngeneic fibrosarcoma cell line (Meth A, provided by A. Berger at the National Cancer Institute, Bethesda, Md.). Experiments were conducted adhering to the principles outlined by the Animal Use Committee of the University of Pennsylvania, in compliance with the Guide for the Care and Use of Laboratory Animals prepared by the Institute of Laboratory and Animal Research, National Research Council. Six to eight week old female Balb-Cl mice were de-haired on the right flank a day prior to injection. Meth A cells were cultured in RPMI 1640 (BioWhittaker, Walkersville, Md.) supplemented with 10% FCS (Hyclone, Logan, Utah) and 2 mM 1-glutamine (BioWhittaker). Samples were grown to 50% confluence in T-150 flasks prior to harvesting by trypsinization (Trypsin-EDTA, Mediatech, Hernden, Va.). Meth A cells were counted and then resuspended in PBS (1×10⁶ cells/mouse) prior to injection. All samples were prepared in PBS to a total volume of 0.5 ml and subcutaneously injected with a 20 gauge needle. Table II lists the corresponding control and treatment groups. The decision to use human-GMCSF over mouse-GMCSF was based on previous investigations indicating efficacy in a mouse tumor model. Prior to flank injection, animals were anesthetized with an intraperitoneal injection of xyloxine (12.5 μg) and ketamine HCl (1.25 mg) in 250 μl of PBS. Animals were sacrificed by CO₂ inhalation when flank tumor burden in the control groups exceeded 1.5 cm in largest diameter. The flanks were then dissected to inspect the tumor burden and/or the microparticles. Dissected tumor and microparticles were measured, weighed, and fixed in formalin for H&E staining.

TABLE II
Mouse Tumor Prevention Experiments Using Meth A Fibrosarcoma
Cells (1 × 10⁶ cells/mouse)*

| | Stimulatory | Cytokine-releasing |
|---|---|---|
| | Microparticle | Microparticle |
| Group | (0.25 mg) | (0.25 mg) |
| Tumor control (n = 8) | — | — |
| Microparticle control | PG-denaturated cd28- | PG-Lys control |
| (n = 8) | control | |
| cd3/cd28-MP alone | PG-cd3/cd28 | PG-Lys-control |
| (n = 8) | | |
| Combinaztion therapy | PG-cd3/cd28 | PG-Lys/GMCSF |
| (n = 8) | | |

*Samples were prepared in PBS with a total volume of 0.5 ml and subcutaneously injected in the right flank using a 20 gauge needle.

A second set of experiment was conducted to evaluate the efficacy of PG-Lys/GMCSF and microparticle size. Microparticles based on PG-Lys/GMCSF were prepared as mentioned above. PG-MP with an average size of 7 μm was isolated by mechanical sieving as mentioned above. The 7 μm PG-cd3/cd28 and PG-Lys/GMCSF microparticles were then prepared as described above. Meth A cells were grown to 50% confluence in T-150 flasks prior to harvesting by trypsinization (Trypsin-EDTA, Mediatech, Hernden, Va.). The cells (1×10⁶ cells/mouse) were counted and resuspended with the 7 um microparticles in PBS prior to injection. The samples were subcutaneously injected with a 20-gauge needle in previously anesthetized mice. Table 1B lists the corresponding control and treatment groups. Animals were sacrificed by CO₂ inhalation when flank tumor burden in the control groups exceeded 1.5 cm in largest diameter. The flanks were then dissected to inspect the tumor burden and/or the microparticles. Dissected tumor and microparticles were measured, weighed, and fixed in formalin for H&E staining.

### EXAMPLE 9

### Characterization of Polyglycolide Microparticles (PG-MP)

After mechanical sieving, the resultant PG-MP were found to have the following physical properties (Table III). The particle size distribution ranged from 32 microns to 96 microns with a volume weighted mean diameter and median diameter of 56 microns, respectively. Potentiometric titration of accessible carboxylic acid groups was 0.3 mmole/g, which corresponds to a zeta potential of −21.87 mV in PBS. Results from ESCA revealed an atomic concentration of 58% carbon and 42% oxygen on the microparticles surface (Tables IV and V). This consisted of carbonyl oxygen, carbonyl ester, and carbon-carbon bonds. SEM analysis of the uncoated microparticles showed a highly textured surface. At higher magnifications, however, the microparticles were noted to have a significant porous structure. This was due to the exothermic nature of the polymerization reaction and removal of entrapped, unreacted monomer. Assay for endotoxin was found to be negative.

TABLE III
Physical Characteristics of Polyglycolide Microparticles (PG-MP).

| Parameter | Measurement | Measurement |
|---|---|---|
| Particle size distribution | 14.58-96.41μ | 2.09-14.58μ |
| Volume weighted mean size | 56.31μ | 7.02μ |
| Median size | 56.20μ | 6.85μ |

| | |
|---|---|
| Accessible carboxylic acid groups | 0.3 mmoles/g |
| Zeta potential | −21.87 mV |

TABLE IV
Electron Spectroscopy for Chemical Analysis (ESCA) of
Polyglycolide (PG) Microparticles*

| | Position | | Atomic | |
|---|---|---|---|---|
| | Binding | | Concentration | |
| Peak | Energy (eV) | Atomic Mass | (%) | Chemical Bond |
| O 1s (1) | 531.400 | 15.99 | 24.73 | C═O |
| O 1s (2) | 529.920 | 15.99 | 17.21 | 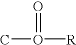 |
| C 1s (1) | 287.024 | 12.011 | 23.27 | C═O |
| C 1s (2) | 284.628 | 12.011 | 24.00 | 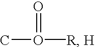 |
| C 1s (3) | 282.811 | 12.011 | 10.34 | C—C |

*Electron spectroscopy for chemical analysis (ESCA) of polyglycolide (PG) microparticles. The atomic concentration of carbon and oxygen were 57% and 43%, respectively. Different chemical bonds produce slight shifts in the positional binding energy which provides additional structural information.

TABLE V
Electron Spectroscopy for Chemical Analysis (ESCA) of Polyglycolide
(PG) Microparticles*

| | Atomic | Atomic | Atomic |
|---|---|---|---|
| | Conc. (%) | Conc. (%) | Conc. (%) |
| Sample | Oxygen | Carbon | Nitrogen |
| PG | 42.91 | 57.01 | — |
| PG/GM-CSF | 46.51 | 52.52 | 0.97 |
| PG/p-lysine | 41.29 | 57.00 | 1.47 |
| PG/p-lys/GM-CSF | 31.12 | 63.16 | 4.20 |
| PG/GM-CSF(PR) | 38.60 | 59.79 | 1.61 |
| PG/p-lys/GM-CSF | 36.31 | 57.91 | 1.80 |
| (PR) | | | |

*Samples were coated with either GM-CSF or poly-l-lysine (p-lys) followed by GM-CSF. Selected samples were also analyzed after GM-CSF release studies were performed. Post-release samples are designated by (PR).

TABLE VI
Electron Spectroscopy for Chemical Analysis (ESCA)*

| Pre-release PG-Lys/GMCSF Microparticle | | | Post-release PG-Lys/GMCSF Microparticle | | |
|---|---|---|---|---|---|
| | Position Binding | Atomic | | Position Binding | Atomic |
| Peak | Energy (eV) | Concentration (%) | Peak | Energy (eV) | Concentration (%) |
| O 1s 1 | 529.85 | 19.39 | O 1s 1 | 529.82 | 19.45 |
| O 1s 2 | 531.49 | 11.93 | O 1s 2 | 531.41 | 13.85 |
| — | — | — | O 1s 3 | 533.16 | 1.33 |
| — | — | — | O 1s 4 | 528.26 | 0.83 |

*This Table describes the binding energy and corresponding atomic concentrations of oxygen on PG-Lys/GM-CSF microparticles before and after GM-CSF release studies. Two additional oxygen peaks were noted on the post-release samples which is consistent with the presence of new carboxylic acid groups produced from surface hydrolysis of the polyester linkages.

### EXAMPLE 10

### Electron Spectroscopy for Chemical Analysis (ESCA) of Surface-Modified PG Microparticles and Control

The presence of nitrogen on the surface, as determined by ESCA, provides information regarding the relative affinity of GM-CSF for the microparticle surface (Table V). PG-MP incubated with GM-CSF only resulted in a marginal increase in the atomic concentration of nitrogen. Incubating PG-MP with poly-1-lysine resulted in a small increase to 1.47% which may reflect a slightly higher adsorption affinity due to a charge neutralization effect. However, pre-adsorbing PG-microparticles with poly-1-lysine followed by the addition of GM-CSF resulted in a nearly 3-fold increase in surface nitrogen. It is believed that the proposed positive shift in zeta potential with the pre-adsorption of poly-1-lysine promoted enhanced adsorption of the negatively charged GM-CSF (pI-5.23). GM-CSF release studies were performed as described below. ESCA done on these samples at the conclusion of the study showed a decrease in surface nitrogen to 1.80% for the PG/poly-lysine/GM-CSF group, but an increase to 1.60% in PG/GM-CSF group. In both cases, the residual nitrogen content reflects irreversible adsorption of either GM-CSF and/or poly-1-lysine. The increase in surface nitrogen was a surprising observation in the PG/GM-CSF group. However, given the microporosity of the microparticles, it is conceived that the poly-1-lysine and GM-CSF can become entrapped within this network. As surface hydrolysis occurs over time, entrapped protein gains exposure to the bulk media and may be released. At the conclusion of the release study, the atomic concentration of nitrogen from ESCA reflects that of irreversibly bound protein on a hydrolyzed MP-surface. Surface degradation is supported by the presence of two additional oxygen peaks as seen by ESCA (Table VI). This represents the carbonyl oxygen and hydroxyl bonds from the carboxylic acid moiety produced by hydrolysis of the polyester linkages.

### EXAMPLE 11

### Anti-cd3/cd28 Microparticles Induce Significant Activation of T Cells

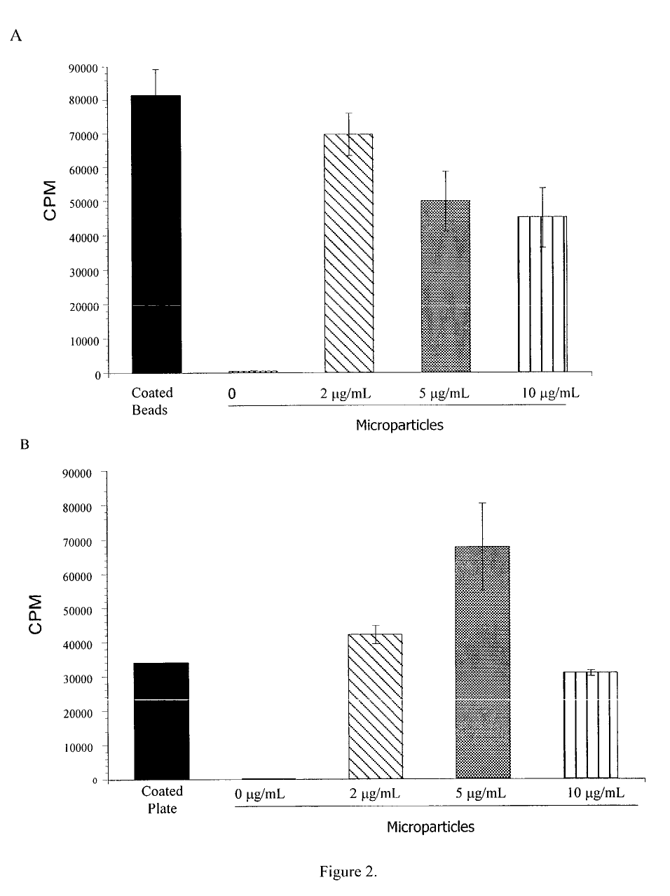

The ability of PG microparticles prepared with either anti-human CD3/CD28 or anti-mouse cd3/cd28 to stimulate resting T cells was tested by measuring ³H-thymidine uptake. FIG. 2 shows that CD3/CD28-MP could induce a significant proliferation of T cells at levels comparable to antibody-conjugated magnetic beads. The proliferative response was antiCD3/CD28-mediated since uncoated microparticles had no effect. The maximum stimulatory effect was noted in the microparticles prepared with an antibody concentration of 2 μg/ml. Thus, data supports the contention that stimulatory and costimulatory immunoglobulins can be immobilized on the MP surface with maintenance of bioactivity. Interestingly, a decrease in proliferation was seen with the 5 μg/ml and 10 μg/ml conditions. It is conceived that these preparation conditions may have had a more profound stimulatory effect on the T cells which occurred at an earlier time point. Alternatively, antibody desorption may have taken place from the MP surface leading to T cell anergy. However, ELISA experiments were unable to detect soluble antibody from supernatants collected after incubating CD3/CD28-MP for one week in PBS at 37° C. FIG. 2B shows that similar levels of proliferation could be achieved in a mouse model. It appears that the 5 μg/ml coating concentration was optimal. Furthermore, we speculate that the decrease in stimulatory effect between the 5 μg/ml and 10 μg/ml conditions may have been due to the day chosen to add ³H-thymidine. Regardless of the preparation conditions, the cd3/cd28-MP were either equivalent or superior to coated cd3/cd28 well plates at stimulating mouse T cells.

Intracellular flow cytometry staining for IL-4 and IFN-γ was evaluated in mouse T cells cultured with either uncoated-MP or anti-cd3/cd28-MP. Cytokine production was significant for IFN-γ and not IL-4. As expected, unstimulated T cells had minimal IFNγ production. However, stimulation with anti-cd3/cd28-MP resulted in more than 50% of T cells producing IFN-γ. Thus, anti-cd3 and anti-cd28 antibodies maintain biologic activity after irreversible adsorption to PG-MP and that the stimulatory/co-stimulatory signal is consistent with a TH1 response.

FIG. 2 illustrates activation of resting human (A) and mouse (B) t-cells by anti-human CD3/CD28-MP and anti-mouse cd3/cd28-MP, respectively. Anti-CD3/CD28 magnetic beads (A) and well plates coated with anti-cd3 and anti-cd28 (B) were used as positive controls. T-cells were incubated in triplicate with either 0.6 mg/well of polyglycolide microparticles (0 μg/ml) as a control or cd3/cd28-bearing microparticles prepared with either 2 μg/ml, 5 μg/ml, or 10 μg/ml of antibody. Proliferation was assessed by pulsing the plates with 2 μCi/well [³H] TdR on day 3 followed by cell harvesting 18 hours later.

### EXAMPLE 12

### Prolonged Release of GM-CSF from PG-Microparticles

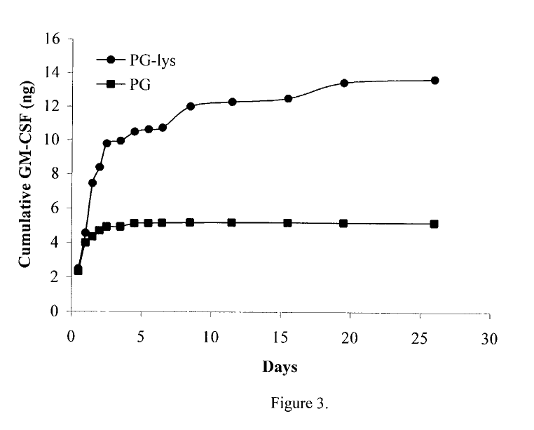

The preceding ESCA data (Table V) indicated that surface charge manipulation via poly-1-lysine (FIG. 1) could be utilized to increase the adsorption affinity of h-GMCSF on the microparticles. It was conceived that irreversible adsorption may be possible as a function of protein size. The release of GM-CSF from the microparticle surface was evaluated by ELISA using thawed supernatants collected over the course of 26 days at 37° C. FIG. 3 shows the GM-CSF release profiles from microparticles prepared from PG and PG-Lys. GM-CSF release from PG microparticles was complete by day four. This represents the extent of reversibly adsorbed cytokine. The presence of irreversibly bound GM-CSF was observed by ESCA (Table V) showing a significant nitrogen peak at the conclusion of the study. Manipulating surface charge with poly-1-lysine not only enhanced GM-CSF adsorption, but also increased the magnitude and duration of GM-CSF release (FIG. 3). Release from PG-Lys was observed for up to 26 days with a three-fold increase in the amount of cytokine released as compared to the PG samples. Nearly 75% of total GM-CSF was released by day seven with the remaining fraction releasing over the next 19 days. The release mechanism may be attributed to relatively stronger GM-CSF interactions with PG-Lys, which may require surface hydrolysis to promote release. Interestingly, the level of irreversibly bound protein was similar to that of the PG-MP. However, this could reflect a combination of poly-1-lysine and/or GM-CSF.

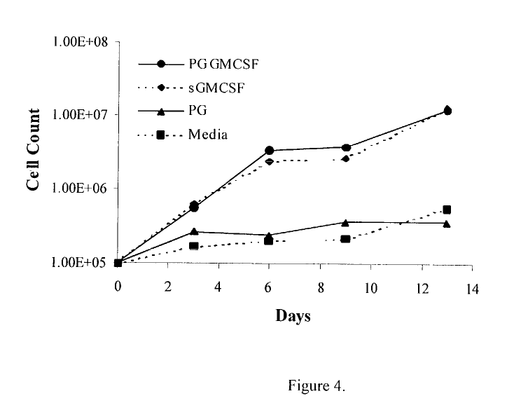

The growth of TF-1 cells showed that the activity of GM-CSF released by the microparticle was equivalent to supplementation with soluble GM-CSF every two days (FIG. 4). A single 10 mg dose of PG-Lys/GMCSF was able to expand the TF-1 cells for the two week culture period. Conditions with uncoated microparticles or media alone demonstrated minimal growth of the TF-1 cells. Thus, we demonstrate that PG microparticles can be used to deliver functionally active cytokines in a prolonged and efficient manner.

FIG. 3 illustrates the release of GM-CSF from GMCSF-MP prepared from PG and PG-Lys. GY-CSF was assayed by ELISA.

FIG. 4 illustrates the Growth of TF-1 cells cultured with GM-CSF-releasing microparticles (●), soluble GM-CSF @ 2 ng/ml (♦), uncoated PG-MP (▴), and plain culture media (▪).

### EXAMPLE 13

### PG-microparticles Prevent Mouse Tumor Implantation

The results of the two tumor prevention experiments are shown in Table VII. Dissection of the flanks showed that the microparticles formed a well circumscribed conglomerate in the subcutaneous tissue. This was seen with all control and treatment groups using the 56 micron microparticles. However, a dramatic loss of mechanical integrity was noted with the 7 micron microparticles. Upon removal, they had a viscoelastic consistency in contrast to the 56 um particles which maintained their shape. This dramatic level of degradation was not surprising given the increased surface area to hydrolyze the polymer chains. Interestingly, the microparticle systems displayed no intrinsic ability to impair tumor implantation. Average tumor sizes of 8.2 mm and 8.6 mm were found in the tumor only and microparticle control groups, respectively. Significant efficacy was observed when Meth A cells were co-injected with either the cd3/cd28-MP or GMCSF-MP alone. Only 5/16 mice developed tumor when Meth A cells were co-injected with the cd3/cd28-MP alone. This corresponded to a nearly 86% reduction in average tumor diameter. Mice receiving GMCSF-MP alone developed tumor in only 2/8 mice. Again, a nearly 86% reduction in average tumor diameter was seen. The most significant effect was seen when the cd3-cd28-MP and GMCSF-MP were combined. There was no evidence of tumor implantation with either the 56 micron (0/16) or 7 micron (0/8) microparticle sizes.

TABLE VII
Results of Mouse Tumor Prevention Experiments Using Meth A Fibrosarcoma
Cells

| | Number | Mice with Tumor | Average Tumor Diameter | |
|---|---|---|---|---|
| Group | of Mice | Present | (mm) | SE (+/−) |
| Tumor only | 16 | 16 | 8.2 | 0.71 |
| Microparticle control | 15 | 15 | 8.6 | 1.10 |
| cd3/cd28-MP aloine | 16 | 5 | 1.2 | 0.46 |
| GMCSF-MP alone | 8 | 2 | 1.0 | .68 |
| Combination group | 16 | 0 | — | — |
| (56 μm particle size) | | | | |
| Combination group | 8 | 0 | — | — |
| (7 μm particle size) | | | | |

Although the present invention has been described in connection with the preferred embodiments, it is to be understood that modifications and variations may be utilized without departing from the principles and scope of the invention, as those skilled in the art will readily claims. Moreover, Applicants hereby disclose all subranges of all ranges disclosed herein. These subranges are also useful in carrying out the present invention.

We claim:

1. A therapeutic vaccine for treating cancer comprising absorbable microparticles/nanoparticles comprising a crystalline absorbable polymer having a heat of fusion of at least about 60 Joules/gram, said microparticles/nanoparticles comprising a surface having at least one bioactive agent immobilized thereon.

2. The therapeutic vaccine set forth in claim 1 wherein the surface of the microparticles/nanoparticles is anion-forming.

3. The therapeutic vaccine set forth in claim 2 wherein the at least one bioactive agent is ionically bound to the surface of the microparticles/nanoparticles.

4. The therapeutic vaccine set forth in claim 2 wherein the crystalline absorbable polymer comprises an acid-bearing polyglycolide.

5. The therapeutic vaccine set forth in claim 4 wherein the acid-bearing polyglycolide comprises an acid-terminated polyglycolide.

6. The therapeutic vaccine set forth in claim 1 wherein the surface of the microparticles/nanoparticles is microporous/nanoporous.

7. The therapeutic vaccine set forth in claim 3 comprising anti-CD3/anti-CD28 and GM-CSF ionically bonded to the surface of the microparticles/nanoparticles.

8. The therapeutic vaccine set forth in claim 4 comprising at least one bioactive agent ionically bonded to the surface of the microparticles/nanoparticles, the at least one bioactive agent selected from the group consisting essentially of anti-CTLA-4, anti-ICOS, 4-1BB ligand, IL-12, IL-18, IL-2, and IFN-gamma.

9. The therapeutic vaccine set forth in claim 1 further comprising a polycationic molecule chemically bonded to the surface of the microparticles/nanoparticles.

10. The therapeutic vaccine set forth in claim 9 wherein the polycationic molecule comprises polylysine.

11. The therapeutic vaccine set forth in claim 9 further comprising anti-CD3/anti-CD28 and GM-CSF bound to the polycationic molecule.

12. The therapeutic vaccine set forth in claim 9 further comprising at least one bioactive agent chemically bonded to the polycationic molecule, the at least one bioactive agent selected from the group consisting essentially of anti-CTLA-4, anti-ICOS, 4-1BB ligand, IL-12, IL-18, IL-2, and IFN-gamma.

13. The therapeutic vaccine set forth in claim 9 wherein the polycationic molecule is ionically bonded to the surface of the microparticles/nanoparticles.

14. The therapeutic vaccine set forth in claim 13 wherein the polycationic molecule is at least partially covalently bonded to the surface of the microparticles/nanoparticles.

15. The therapeutic vaccine set forth in claim 1 wherein the surface of the microparticles/nanoparticles is cation-forming.

16. The therapeutic vaccine set forth in claim 15 wherein the crystalline absorbable polymer comprises an amine-bearing polyester.

17. The therapeutic vaccine set forth in claim 15 comprising anti-CD3/anti-CD28 and GM-CSF ionically bonded to the surface of the microparticles/nanoparticles.

18. The therapeutic vaccine set forth in claim 15 comprising at least one bioactive agent ionically bonded to the surface of the microparticles/nanoparticles, the at least one bioactive agent selected from the group consisting essentially of anti-CTLA-4, anti-ICOS, 4-1BB ligand, IL-12, IL-18, IL-2, and IFN-gamma.
